(19) United States
(12) Patent Application Publication
(10) Pub. No.: US 2002/0064505 A1
(43) Pub. Date: May 30, 2002

(54) Oral anti-odor compositions

(76) Inventor: Melvyn Nevo Rosenberg, Ramat-Gan (IL)

Correspondence Address: BAKER & BOTTS, 30 ROCKEFELLER PLAZA, NEW YORK, NY, 10112

(21) Appl. No.: 09/962,561

(22) Filed: Sep. 24, 2001

Related U.S. Application Data
(63) Continuation of application No. PCT/IL00/00183, filed on Mar. 24, 2000.

(51) Int. Cl. A61K 9/68; A61K 7/16; A61K 7/26

(52) U.S. Cl. 424/49; 424/58

(57) ABSTRACT

The invention is directed to a composition comprising a higher alcohol selected from 1-nonanol, 1-decanol and 1-undecanol, or mixtures thereof and taste-masking additives, or a lactone selected from gamma-methyl decalactone, gamma-decalactone, gamma-undecalactone and gamma dodecalactone or mixtures thereof for use as an oral anti-odor preparation. The invention further provides anti-odor toothpaste, mouthwash, mouth drops, candies and other anti-odor preparations for oral use.

### FIELD OF THE INVENTION

The present invention is concerned with compositions containing higher alcohols, particularly those having chain length C9 and greater, and their formulation for use as anti-odor agents, particularly for oral use.

### BACKGROUND OF THE INVENTION

Bacterial products and activity are implicated in many oral diseases, such as periodontal disease and caries, as well as in the production of unpleasant odors in the mouth. Because of the multitude of bacterial species found in the oral cavity, there is a need for effective non-specific anti-microbial agents, with a broad spectrum of activity. Such agents may be incorporated into a number of different preparations, including: mouthwashes, mouthsprays, toothpastes and other dentifrices, chewing gum, candies and related products.

For many years, short-chain alkyl alcohols such as ethanol and 2-propanol have been used clinically and domestically as surface-active anti-microbial agents. In addition, there have been a few reports that describe inhibitory effects of individual higher alcohols (chain length up to C16) on the growth of certain bacteria (particularly gram positive species), bacterial spores and fungi [see Kato & Shibasaki, Bokin Bobai.8: 325 (1980); Gershon & Shanks, J. Pharm. Sci. 69: 381 (1980); Yasuda-Yasaki et al., Spores 7: 113 (1978)]. In addition, JP 62-230712 (assigned to Nichibai Boeki KK) discloses the use of higher alcohols in the management of dental caries, a condition in which the gram positive bacteria Streptococcus mutants is believed to play a major pathogenic role.

The current invention concerns higher alcohols (C8-C14), used singly or in combinations, which are highly effective as antimicrobial agents against a very broad spectrum of bacteria and yeasts. The invention also provides the formulation and use of these higher alcohols as anti-odor agents. While the mechanism of the anti-odor effect is not precisely known, it may be related to a shift in microbial populations at the treatment site. It would appear that it is particularly the gram negative resident species of the oral cavity that are implicated in the production of bad breath. The hitherto unreported highly efficient inhibition of these species by the higher alcohols is thus one of the key features of this invention.

The higher alcohols have not previously been used as the primary components of anti-odor products for oral use. This may be, in part, because of their characteristic, strong, citrus-like odors and flavors. In the present invention, however, it has been surprisingly found that low concentrations of the higher alcohols have an acceptable flavor and odor. Thus, when used at appropriate concentrations, the higher alcohols, in addition to their primary antimicrobial and anti-odor effects, also confer positive organoleptic properties on oral anti-odor agents containing said higher alcohols. In another aspect of the invention, the abovementioned problematic strong odors and flavors contributed by the higher alcohols when used at higher concentrations is overcome by the use of novel combinations of taste-masking additives. p It is a primary object of this invention to provide the effective oral anti-odor preparations.

It is another object of this invention to provide oral anti-odor compositions having a pleasant taste, in order to improve patient compliance.

It is yet another purpose of this invention to provide the aforementioned oral anti-odor compositions in a variety of forms including mouthwash, chewing gum, toothpaste mouth drops and candies.

Other objects and advantages of the invention will become apparent as the description proceeds.

### SUMMARY OF THE INVENTION

It has now been surprisingly found that the higher alkyl alcohols are highly effective as oral anti-odor agents, displaying an unexpectedly high level of activity against gram negative bacteria, which have been reported to be the main causative agents in the development of bad breath. This stands in sharp contrast with what has been disclosed in the prior art, where the higher alcohols are described as possessing antibacterial effects primarily on gram positive organisms. It has also been found that the intrinsic flavour of some oral preparations containing higher alcohols, which is sometimes a factor reducing patient compliance, may be adequately masked by including taste-masking additives in said oral preparations, without loss of the antimicrobial and anti-odor properties of said higher alcohols.

The invention is primarily directed to an anti-odor composition comprising a higher alcohol together with taste-masking additives, for oral use. Particularly, the invention is directed to a composition comprising a higher alcohol selected from 1-nonanol, 1-decanol and 1-undecanol, or mixtures thereof and taste-masking additives, as an oral anti-odor preparation.

According to one preferred embodiment of the invention, the higher alcohol is 1-nonanol. According to another preferred embodiment, the higher alcohol used 1-decanol. In another preferred embodiment, the higher alcohol is 1-undecanol.

Although any suitable taste-masking additives may be used in conjunction with the above-mentioned higher alcohols, preferred taste-masking additives include nerol, citral and menthol, mint, spearmint and peppermint oils, or other mint flavors, or mixtures thereof.

In another aspect, it has now been found that glycerol is a particularly suitable solvent for the purpose of the present invention. Glycerol is particularly convenient, since it is a GRAS substance, and therefore can be used in food and food-related products without any limitation. A further advantage of the use of glycerol is that it is suitable for use in the preparation of soft-centred candies and chewing gums.

In one preferred embodiment, glycerol is incorporated into mouth drops.

The invention provides an oral anti-odor composition comprising a higher alcohol together with taste-masking additives, provided in a glycerol based carrier.

In another aspect, the invention is directed to an anti-odor toothpaste comprising a higher alcohol selected from 1-nonanol, 1-decanol and 1-undecanol, or mixtures thereof, together with one or more taste-masking additives.

According to one preferred embodiment of the invention, the higher alcohol is 1-nonanol. According to another preferred embodiment, the higher alcohol used 1-decanol. In another preferred embodiment, the higher alcohol is 1-undecanol.

Although any suitable taste-masking additives may be used in conjunction with the above-mentioned higher alcohols, preferred taste-masking additives include nerol, citral and menthol, mint, spearmint and peppermint oils, or other mint flavors, or mixtures thereof.

In a further aspect, the invention is directed to an anti-odor candy comprising a higher alcohol selected from 1-nonanol, 1-decanol and 1-undecanol, or mixtures thereof, together with one or more taste-masking additives.

The above-mentioned anti-odor candy may be in the form of any of the commonly found types of confection. According to one preferred embodiment, the anti-odor candy is a chocolate candy. In another preferred embodiment the anti-odor candy is in the form of a chewable candy. In a further preferred embodiment, the anti-odor candy is in the form of chewing gum. The anti-odor chewing gum of the invention is not limited to any particular form or type of gum, and includes all of the commonly-known types including gum formed into chiclet-type preparations, strips, pellets, spheres, and so on.

In some instances, it is desirable to provide the active compositions of the invention in sequestered form, so that they are delivered to the appropriate site in the oral cavity, and at the desired time. Illustrative examples of suitable sequestration methods include, e.g., microencapsulation, dispersion in a solid matrix, etc. Many such methods are known in the art, and will be apparent to the skilled person.

One such example is a dragé-covered chewing gum, which incorporates the active composition of the invention in the gum itself. Typically, the dragé will not comprise the composition of the invention, which will be liberated in to the oral cavity by the chewing action.

In another preferred embodiment, the anti-odor candy of the invention is a slowly dissolving product.

While the slowly dissolving candy may take many forms, in a preferred embodiment, it is a pastille. According to another preferred embodiment, the slowly dissolving product is a hard candy. In yet another preferred embodiment, the hard candy has a soft center.

All of the abovementioned anti-odor candies may comprise any of the aforementioned higher alcohols. According to a preferred embodiment, however, the higher alcohol is 1-nonanol. According to another preferred embodiment, the higher alcohol is 1-decanol. In a further preferred embodiment, the higher alcohol is 1-undecanol. Although each of the above-mentioned anti-odor candies may contain any suitable taste-masking additives, in a preferred embodiment, the taste-masking additives comprise nerol, citral and menthol, mint, spearmint and peppermint oils, or other mint flavors.

The anti-odor candies of the invention are not limited to the examples mentioned hereinabove, or to the formulations listed hereinbelow. Rather, the invention is directed to anti-odor candies in all of the forms of candy that are known in the art.

In another aspect, the invention is directed to an anti-odor mouthwash comprising a higher alcohol selected from 1-nonanol, 1-decanol and 1-undecanol, or mixtures thereof, together with taste-masking additives. According to one preferred embodiment of the invention, the higher alcohol is 1-nonanol. According to another preferred embodiment, the higher alcohol used 1-decanol. In another preferred embodiment, the higher alcohol is 1-undecanol.

Although the anti-odor mouthwash may contain any suitable taste-masking additives, preferred taste-masking additives include nerol, citral and menthol, mint, spearmint and peppermint oils, or other mint flavors, or mixtures thereof.

The invention also encompasses an anti-odor mouthspray comprising a higher alcohol selected from 1-nonanol, 1-decanol and 1-undecanol, or mixtures thereof, together with taste-masking additives.

The invention is also directed to anti-odor mouthdrops comprising a higher alcohol selected from 1-nonanol, 1-decanol and 1-undecanol, or mixtures thereof, together with taste-masking additives.

In a further aspect, the invention is directed to an anti-odor cigarette, comprising a higher alcohol selected from 1-nonanol, 1-decanol and 1-undecanol, or mixtures thereof.

The invention further makes provision for a toothpick that is coated or impregnated with a composition comprising a higher alcohol selected from among 1-nonanol, 1-decanol and 1-undecanol, or mixtures thereof, together with taste-masking additives.

In a further aspect, the invention is directed to a dental floss yarn that is coated or impregnated with a composition comprising a higher alcohol selected from among 1-nonanol, 1-decanol and 1-undecanol, or mixtures thereof, together with taste-masking additives.

In another aspect, the invention relates to an anti-odor composition adapted be brought into contact with the oro-pharynx and posterior portion of the oral cavity, comprising a higher alcohol selected from 1-nonanol, 1-decanol and 1-undecanol, or mixtures thereof, together with one or more taste-masking additives.

The invention further provides an anti-odor paste adapted to be applied to the tongue and to the teeth, comprising a higher alcohol selected from 1-nonanol, 1-decanol and 1-undecanol, or mixtures thereof, together with one or more taste-masking additives.

The invention also provides an anti-odor paste adapted to be supplied to the teeth and to be gargled, comprising a higher alcohol selected from 1-nonanol, 1-decanol and 1-undecanol, or mixtures thereof, together with one or more taste-masking additives.

In another aspect, the invention provides an oral anti-odor composition comprising a lactone selected from gamma-methyl decalactone, gamma-decalactone, gamma-undecalactone and gamma-dodecalactone, or mixtures thereof. In a preferred embodiment of the invention, these lactone-containing oral anti-odor compositions further comprise higher alcohols selected from 1-nonanol, 1-decanol and 1-undecanol, or mixtures thereof. In a more preferred embodiment of the invention, the lactone present in these oral anti-odor compositions is gamma-undecalactone.

The invention also encompasses the use of a lactone as a synergistic additive in an oral anti-odor composition.

All the above and other characteristics and advantages of the invention will be further understood from the following illustrative and non-limitative examples of preferred embodiments thereof.

### BRIEF DESCRIPTION OF THE DRAWINGS

The present invention will be more clearly understood from the detailed description of the preferred embodiments and from the attached drawings in which:

FIG. 1 is a graph demonstrating the effect of chewing gum containing Breathanol™ on the level of whole mouth malodor, as measured by Odor Judge 1.

FIG. 2 is a graph demonstrating the effect of chewing gum containing Breathanol™ on the level of whole mouth malodor, as measured by Odor Judge 2.

FIG. 3 is a graph depicting the effect of chewing gum containing Breathanol™ on the level of whole mouth malodor. The data shown on the graph are mean values obtained from both Judges 1 and 2, after correcting for the difference between them at time zero.

FIG. 4 is a graph demonstrating the effect of chewing gum containing Breathanol™ on the level of tongue posterior malodor, as measured by Odor Judge 1.

FIG. 5 is a graph demonstrating the effect of chewing gum containing Breathanol™ on the level of tongue posterior malodor, as measured by Odor Judge 2.

FIG. 6 is a graph demonstrating the effect of chewing gum containing Breathanol™ on the level of tongue posterior malodor, as assessed by the experimental subjects themselves.

### DETAILED DESCRIPTION OF PREFERRED EMBODIMENTS

### EXAMPLES

The following compositions and preparations were used in the examples given hereinbelow:

| 1. Breathanol ™ | |
|---|---|
| nerol | 1 part |
| citral | 1 part |
| 1-nonanol | 1 part |
| peppermint oil | 2 parts |
| 2. Decarboxylase Medium | |
| peptone | 0.5% |
| yeast extract | 0.3% |
| dextrose | 0.1% |
| bromocresol purple | 0.07% |

### Example 1

### Inhibition of in vitro Odor Formation by Toothpaste Containing Breathanol™

A series of test tubes were prepared, each containing:

a) 200 μl of freshly-taken morning saliva;

b) 10-1000 μl of a Breathanol-containing toothpaste that was diluted 1:5 with decarboxylase medium (concentration of Breathanol in the toothpaste before dilution is 1%), and

c) decarboxylase medium, to bring the final volume of the mixture up to 5 ml.

The tubes were incubated at 37° C. for three days. Following incubation, the odor was determined by a panel of odor judges, using the following semi-quantitative scale

0=no odor

1=very slight odor

2=weak but noticeable odor

3=medium odor

4=foul odor

5=extremely foul odor

The results (Table I) show that a reduction in odor formation is seen with all dilutions of the Breathanol-containing toothpaste (compared with no-toothpaste control), and that this anti-odor effect is dose related.

TABLE I

| Volume of | | | | | | |
|---|---|---|---|---|---|---|
| diluted | 1-nonanol | | | | | |
| toothpaste | concentration | | ODOR SCORES | | | |
| (μl) | ppm | % w/w | Judge 1 | Judge 2 | Judge 3 | Mean |
| 10 | 0.8 | 0.00008 | 3 | 4 | 1.25 | 2.75 |
| 25 | 2 | 0.0002 | 2 | 3 | 1.5 | 2.17 |
| 50 | 4 | 0.0004 | 2 | 2.5 | 0.5 | 1.67 |
| 125 | 10 | 0.001 | 1 | 1 | 1 | 1 |
| 250 | 20 | 0.002 | 0.5 | 1 | 0 | 0.5 |
| 500 | 40 | 0.004 | 0 | 1 | 0.75 | 0.58 |
| 750 | 60 | 0.006 | 0 | 1 | 1 | 0.67 |
| 1000 | 80 | 0.008 | 0 | 0 | 0.5 | 0.17 |
| Control | 0 | 0 | 4 | 4.5 | 4 | 4.17 |

### Example 2

### Clinical Study of Inhibition of Odor Formation by Mouthwash Containing Breathanol

The ability of Breathanol to reduce odor was tested in the following clinical study. Subjects (N=51; mean age 24.5 years) were recruited from among those who had previously volunteered for similar studies. Subjects were remunerated for their time. The criteria for exclusion from the study were: taking antibiotics within one month prior to the study, smokers, partial or complete denture wearers. Participants were asked to refrain from eating or drinking 2 hours prior to measurements. Initially, subjects were tested for malodor-related parameters (as described further). They were then given the mouthwash (17 ml; active, containing 1% Breathanol, or placebo), and were asked to swish and gargle twice for 30 seconds with a one minute interval. They were reexamined 1.5 and 3 hours following use.

Subjects were assessed for oral malodor-related parameters, including (i) whole mouth odor as measured by three independent judges on a scale of 0-5; (ii) tongue dorsum posterior odor using the spoon test; and (iii) volatile sulphide levels using the model 1170 sulphide monitor (Interscan Corp., Chatsworth Calif.).

All measurements were made prior to rinsing (time zero), and at 1.5 and 3 hours post-rinsing. Results were analyzed using analysis of variance (ANOVA) and, when necessary, analysis of co-variance (ANCOVA) with time zero as covariate.

Measurements

Organoleptic measurements.

Organoleptic measurements were carried out throughout the study by one experienced and two inexperienced judges whose scores have been compared to other judge's scores and measurement techniques in previous studies.

Organoleptic measurements were made, based on the whole mouth expirate, as well as odor assessment from the posterior of the tongue dorsum. For whole mouth malodor, following a three hour fast, subjects were instructed to exhale briefly through the mouth, at a distance of ca. 10 cm from the nose of the judge. For assessment of the tongue posterior dorsum, a sample was obtained by mild scraping with a plastic spoon. After 5 seconds, the odor judges and the subjects themselves smelled the odor at a distance of ca. 5 cm from the spoon. Results of the two malodor assessments were rated on a semi-integer scale of −5 to 5 as follows:

−5 extremely pleasant odor

−4 very pleasant odor

−3 moderately pleasant odor

−2 slight, but noticeable pleasant odor

−1 barely noticeable pleasant odor

0 no appreciable odor

1 barely noticeable unpleasant odor

2 slight, but clearly noticeable unpleasant odor

3 moderate unpleasant odor

4 strong unpleasant odor

5 extremely foul odor

Volatile Sulphur Compounds (VSC)

VSC of intraoral headspace was measured using the Interscan 1170 monitor. Quantitative measurement of volatile sulphides were carried out using the Interscan 1170 monitor (Interscan Corporation, Chatsworth, Calif.), 1 ppm full-scale deflection. Volunteers were asked to refrain from talking for 5 minutes prior to measurement. The monitor was zeroed on ambient air, and measurement performed by inserting a disposable ¼″ plastic straw approximately 4 cm into the oral cavity. The volunteer was asked to breathe through his/her nose during measurement. Results were recorded as peak ppb sulphide equivalents.

Microbial Counts

Unstimulated whole saliva was collected and diluted in saline. Plating was performed on blood agar. Plates were incubated aerobically at 37° C. for 24 hours.

Statistical Evaluations

Comparison between the various rounds of the study was carried out using ANOVA or ANCOVA. Treatment effects were compared using the t-test with the Bonferroni correction.

Results and Discussion

Organoleptic assessments of whole mouth odor made by the three judges are summarized in Table II. In all cases, much larger reductions were observed in the scores of the experimental, as compared with control, subject groups. In the case of the experienced judge, the decrease in the scores of the active mouthwash group was highly significant as compared with placebo scores (p<0.0005, ANCOVA).

Similar results were obtained for odor deriving from the back of the tongue, and are shown in Table III.

TABLE II

| | Baseline | 90 Minutes | 180 Minutes |
|---|---|---|---|
| Experimental | 1.9 +/− 0.9 | 1.2 +/− 1.07 | 1.0 +/− 1.23^{a} |
| group: | | | |
| experienced | | | |
| judge | | | |
| Control group: | 1.68 +/− 1.079 | 1.64 +/− 0.98 | 1.692 +/− 0.991 |
| experienced | | | |
| judge | | | |
| Experimental | 2.14/−0.698 | 1.660 +/− 0.770 | 1.540 +/− 0.780 |
| group: mean | | | |
| for three | | | |
| judges | | | |
| Control group: | 1.71 +/− 0.714 | 1.583 +/− 0.775 | 1.577 +/− 0.799 |
| mean for three | | | |
| judges | | | |

^{a}Experimental group significantly different from control group (p = 0.0308, t-test)

TABLE III

| | Baseline | 90 Minutes | 180 Minutes |
|---|---|---|---|
| Experimental | 2.400 +/− 0.707 | 1.958 +/− 0.834 | 1.593 +/− 0.649 |
| group: mean | | | |
| for 3 judges | | | |
| Control group: | 2.455 +/− 0.656 | 2.071 +/− 0.488 | 1.917 +/− 0.552 |
| mean for 3 | | | |
| judges | | | |
| Experimental | 1.740 +/− 1.347 | 1.750 +/− 1.445 | 2.240 +/− 1.091 |
| group: self | | | |
| assessment | | | |
| Control group: | 2269 +/− 1.505 | 2.577 +/− 1.172 | 2.846 +/− 1.384 |
| self assessment | | | |

Statistical significance:
Judged results: experimental group significantly different from control group (p = 0.0135; t-test);
Self assessment: experimental group significantly different from control group (p = 0.0304; t-test).

The results of the sulphide assay and of the microbial count measurements are shown in Table IV. The results for the sulphide assay demonstrate that for the expermental group, there was a significant decrease in salivary sulphide content at the 180 minute time point (p=0.0001; ANOVA).

Similarly, the microbial count on blood agar was significantly reduced by the treatment at the 180 minute time point (p=0.0493; ANOVA).

TABLE IV

| Tested | | | |
|---|---|---|---|
| Parameter | Baseline | 90 minutes | 180 minutes |
| Sulphide | 5.151 +/− 0.234 | 4.915 +/− 0.095 | 4.825 +/− 0.087 |
| levels - | | | |
| Experimental | | | |
| Sulphide | 5.060 +/− 0.245 | 5.043 +/− 0.205 | 4.971 +/− 0.276 |
| levels - | | | |
| Control | | | |
| Microbial | 7.001 +/− 0.926 | 5.996 +/− 0.919 | 6.804 +/− 1.203 |
| counts - | | | |
| Experimental | | | |
| Microbial | 6.231 +/− 0.827 | 6.184 +/− 0.825 | 6.771 +/− 0.873 |
| counts - | | | |
| Control | | | |

### Example 3

### Anti-Malodor Properties of Chewing Gum Containing Breathanol™

The anti-malodor properties of chewing gum containing 0.14% Breathanol™ were examined in a group of 64 healthy volunteers. The parameters measured in these subjects were:

(A) Whole mouth odor measured by two judges.

For whole mouth malodor, following a three hour fast, subjects were instructed to exhale briefly through the mouth, at a distance of ca. 10 cm from the nose of the judge. The results of the malodor assessments were rated on a scale of −5 to 5 as described hereinabove in Example 2.

(B) Odor of sample obtained from the posterior tongue dorsum, by means of a spoon (“spoon test”);

For assessment of the tongue posterior dorsum, a sample was obtained by mild scraping with a plastic spoon. Following 5 seconds, the odor judges and the subjects themselves smelled the odor at a distance of ca. 5 cm from the spoon. The results of the malodor assessments were rated on a scale of −5 to 5 as described hereinabove in Example 2.

Subjects were assigned randomly to one of the following three treatment groups:

(i) chewing gum containing 0.14% Breathanol™;

(ii) the same chewing gum with no Breathanol™;

(iii) no chewing gum control.

Subjects were instructed to chew two pieces of chewing gum for 15 minutes. Measurements were made prior to treatment (time zero), at 1.5 and 3 hours.

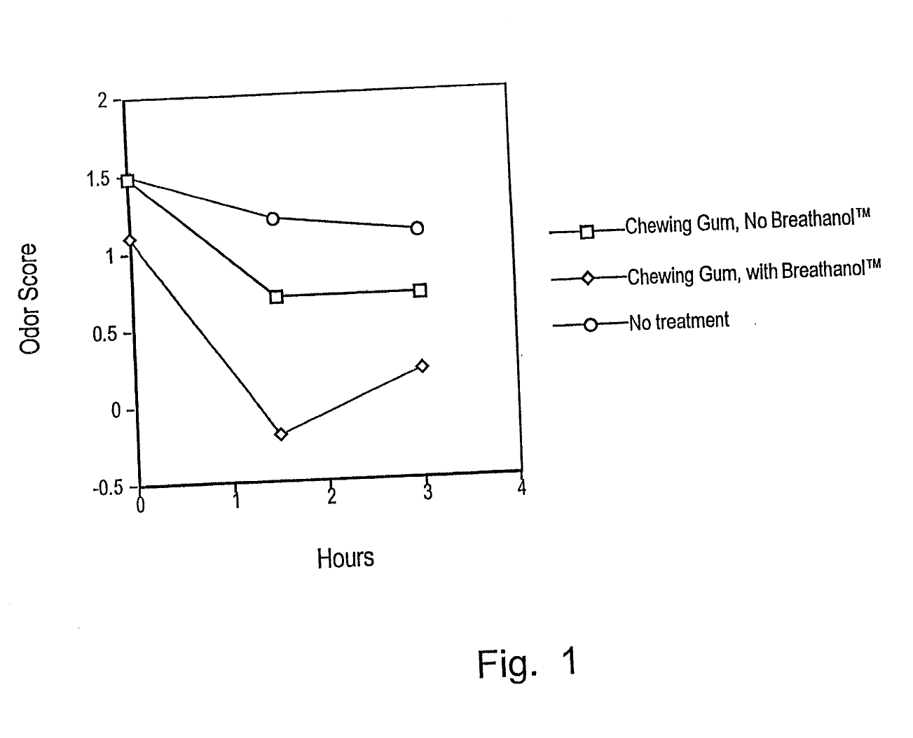

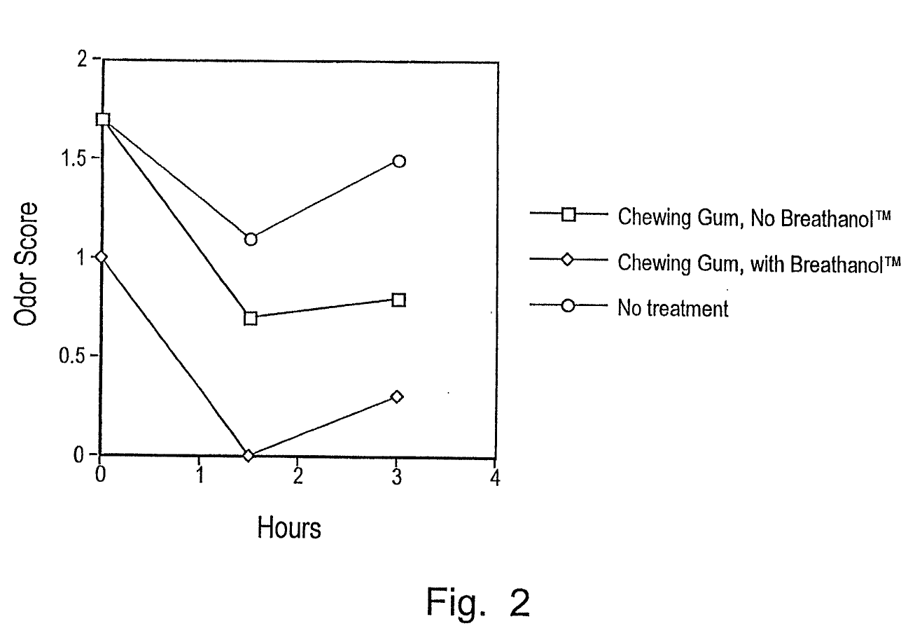

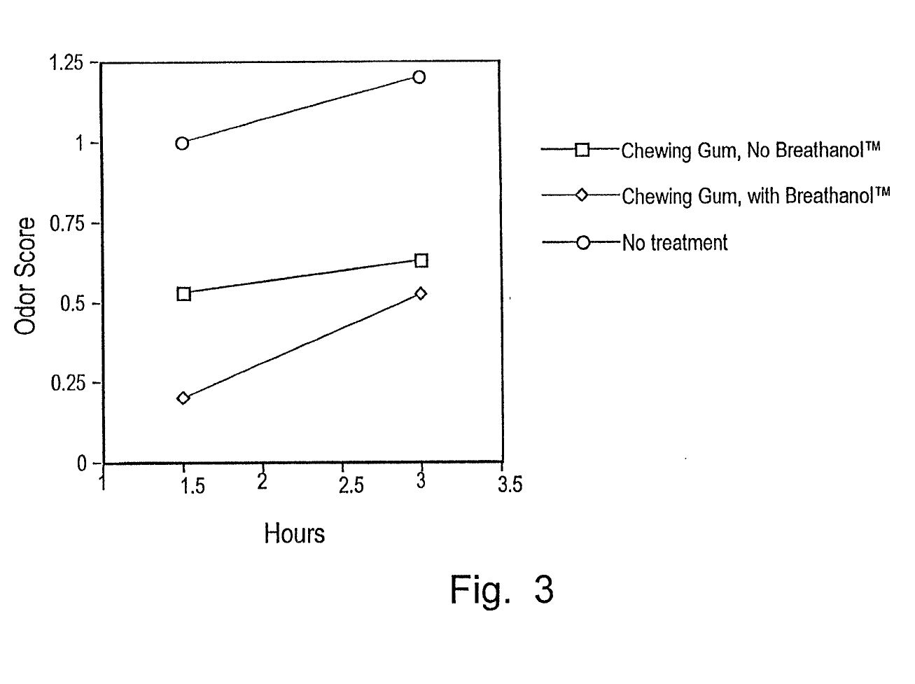

The results for whole mouth odor assessments are given in Table V. The results for Judge 1 are shown graphically in FIG. 1, and those for Judge 2, in FIG. 2. FIG. 3 presents the mean results (for both of the odor judges) after correcting for inter-judge differences at time 0. In each of these graphs, the results for chewing gum with Breathanol™ are depicted by diamond shaped data points, those for chewing gum without Breathanol™ are represented by square datapoints, and the no-treatment control by circles.

TABLE V

| Whole mouth | Chewing gum | Chewing gum | |
|---|---|---|---|
| odor | without | with | No Chewing |
| assessment | Breathanol ™ | Breathanol ™ | gum |
| Judge 1 | 1.48 ± 0.80 | 1.1 ± 1.0 | 1.5 ± 1.0 |
| Time 0 | | | |
| Judge 1 | 0.7 ± 1.5 | −0.2 ± 1.1 | 1.2 ± 1.5 |
| Time 1.5 hours | | | |
| Judge 1 | 0.7 ± 1.3 | 0.2 ± 1.4 | 1.1 ± 1.4 |
| Time 3 hours | | | |
| Judge 2 | 1.7 ± 1.0 | 1.0 ± 1.1 | 1.7 ± 1.0 |
| Time 0 | | | |
| Judge 2 | 0.7 ± 1.3 | 0.0 ± 1.4 | 1.1 ± 1.3 |
| Time 1.5 hours | | | |
| Judge 2 | 0.8 ± 1.5 | 0.3 ± 1.1 | 1.5 ± 1.4 |
| Time 3 hours | | | |

It can be seen from these results that the subjects using the chewing gum containing Breathanol™ gave significantly lower whole mouth odor scores at 1.5 hours than either those using the chewing gum containing Breathanol™ or the untreated subjects. At 3 hours, although the difference between the two chewing gum groups was much less, the group using gum containing Breathanol™ still gave the lowest whole mouth odor score.

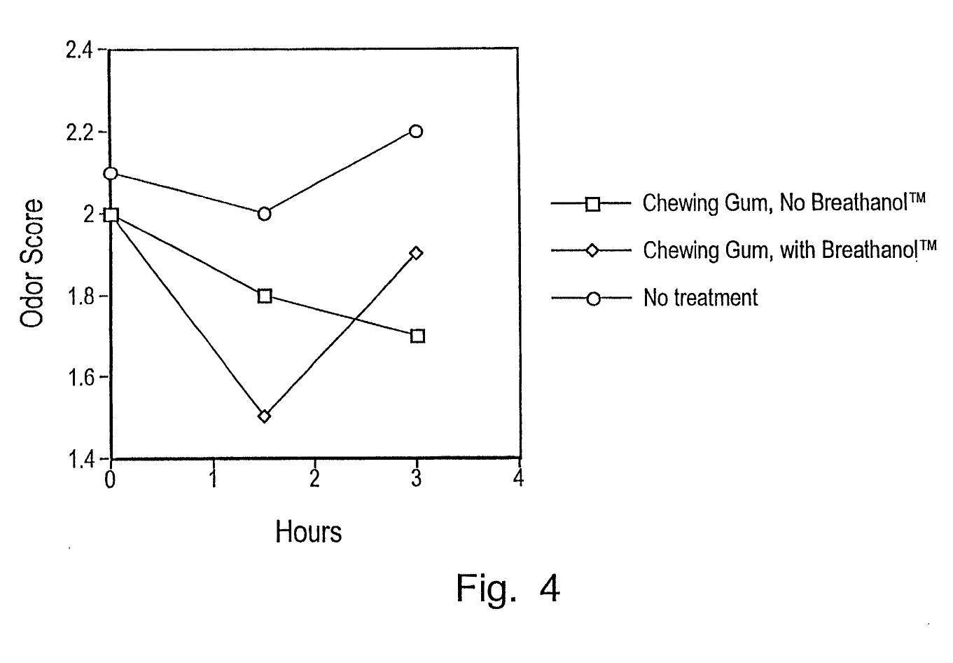

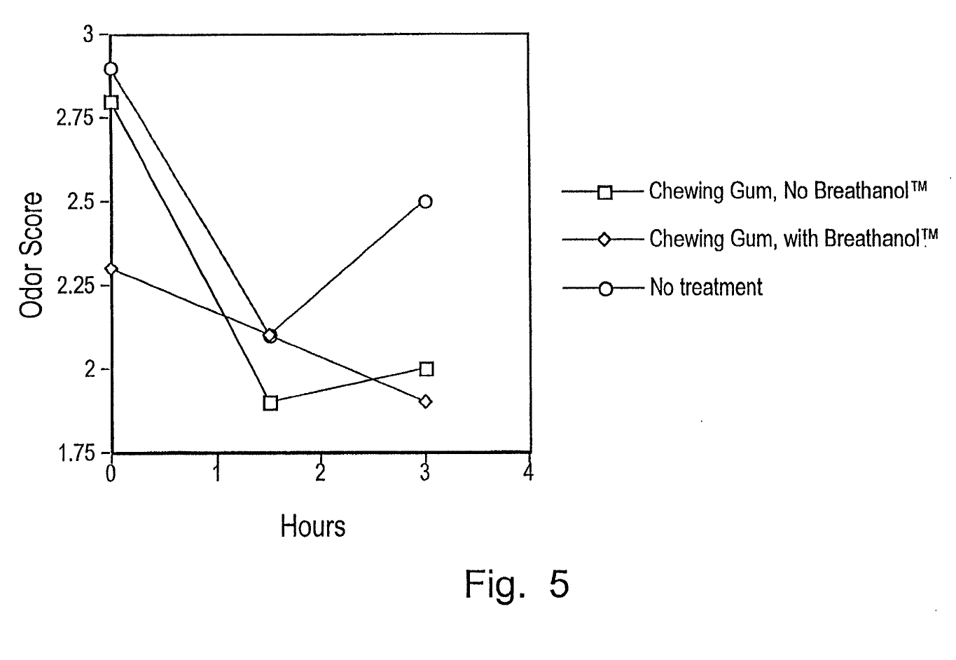

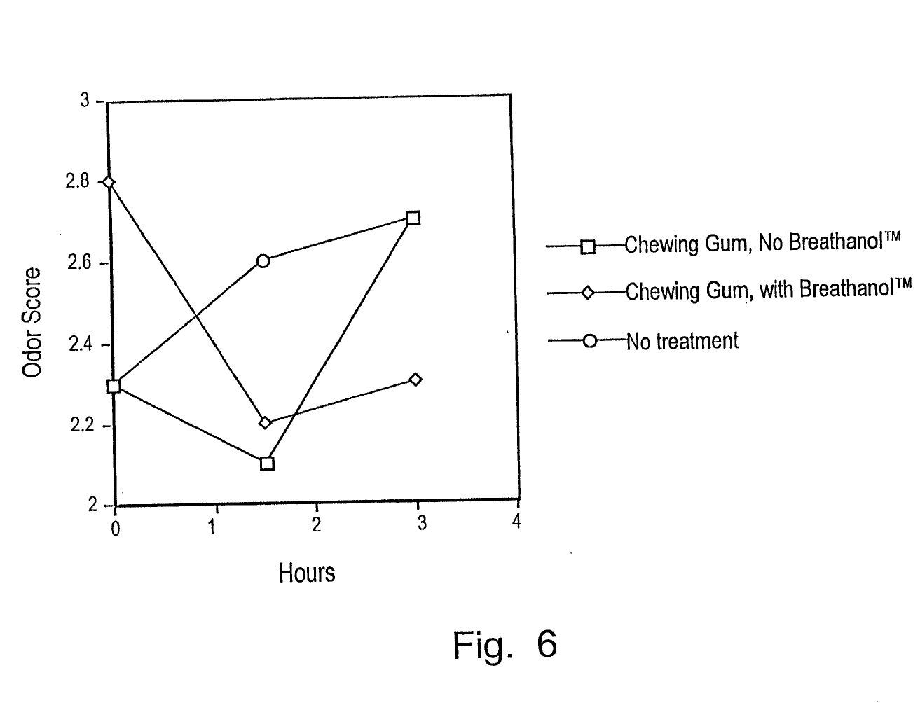

The results for the tongue posterior (spoon test) odor assessments are given in Table VI. The results for Judge 1 are shown graphically in FIG. 4, and those for Judge 2, in FIG. 5. FIG. 6 presents the mean results obtained by self-assessment on the part of the subjects themselves. In each of these graphs, the results for chewing gum with Breathanol™ are depicted by diamond shaped data points, those for chewing gum without Breathanol™ are represented by square datapoints, and the no-treatment control by circles.

TABLE VI

| Tongue | Chewing gum | Chewing gum | |
|---|---|---|---|
| posterior odor | without | with | No Chewing |
| assessment | Breathanol ™ | Breathanol ™ | gum |
| Judge 1 | 2.0 ± 0.9 | 2.0 ± 0.8 | 2.1 ± 1.0 |
| Time 0 | | | |
| Judge 1 | 1.8 ± 0.9 | 1.5 ± 0.7 | 2.0 ± 0.9 |
| Time 1.5 hours | | | |
| Judge 1 | 1.7 ± 0.8 | 1.9 ± 1.1 | 2.2 ± 0.7 |
| Time 3 hours | | | |
| Judge 2 | 2.8 ± 1.0 | 2.3 ± 0.8 | 2.9 ± 0.9 |
| Time 0 | | | |
| Judge 2 | 1.9 ± 1.2 | 2.1 ± 1.0 | 2.1 ± 1.2 |
| Time 1.5 hours | | | |
| Judge 2 | 2.0 ± 1.2 | 1.9 ± 1.1 | 2.5 ± 1.1 |
| Time 3 hours | | | |
| Self assessment | 2.3 ± 0.9 | 2.8 ± 0.9 | 2.3 ± 1.0 |
| Time 0 | | | |
| Self assessment | 2.1 ± 1.1 | 2.2 ± 0.8 | 2.6 ± 1.2 |
| Time 1.5 hours | | | |
| Self assessment | 2.7 ± 1.3 | 2.3 ± 1.0 | 2.7 ± 1.1 |
| Time 3 hours | | | |

It can be seen from these results that the subjects using the chewing gum containing Breathanol™ gave lower tongue posterior odor scores at 3 hours than either those using the chewing gum containing Breathanol™ or the untreated subjects.

### Example 4

### Anti-Malodor Properties of Mouth Drops Containing Breathanol™

The anti-malodor properties of mouth drops containing Breathanol™ were examined in a group of 38 healthy volunteers. The parameters measured in these subjects were:

(A) Whole mouth odor measured by two judges. Measurements were made essentially as described hereinabove in Example 3.

(B) Tongue dorsum posterior odor using the spoon test estimated by both the judges and the subjects themselves. Measurements were made essentially as described hereinabove in Example 3.

(C) Volatile sulphide levels. The method of measurement was essentially the same as that described hereinabove, in Example 2.

(D) A solid phase color test.

Paper disks (3 mm Whatman chromatography paper) were prepared as follows: X-GAL (5 bromo 4 chloro 3 indolyl b D galactopyranozide; 10 ml, 20 mg/ml in Dimethyl formamide) and IPTG (Isopropyl thiogalactopyranozide; 10 ml, 5 mg/ml in distilled water) were applied to each paper disk. Following 24 h incubation at 37° C., X-GAL and IPTG were added again to the paper disks as described above, and the disks were allowed to dry in the incubator for 24 h incubation at 37° C. A saliva sample of 20 ml was applied on each OK2KS disk, and the color intensity was scored, following 10 minutes of incubation at room temperature. The results were rated by one judge on a scale of 0-5, as follows: 0—no appreciable color (white); 1—barely noticeable color; 2—slight but noticeable blue color; 3—moderate blue color; 4—strong blue color; 5—very strong blue color.

Subjects were assigned randomly to one of the two following groups:

(i) mouth drops containing 0.14% Breathanol™;

(ii) the same mouth drops with no Breathanol™.

Five droplets were applied to the center of the tongue dorsum on the evening of the first day, morning, noon and night of day 2 and morning and noon of day 3. Measurements were made prior to treatment (time zero), and at least three hours following the application on noon of day 3. Results were analyzed using ANOVA, and when necessary, ANCOVA with time zero as covariate.

The results of these measurements are summarized in Table VII, in which the data are presented as adjusted means. The statistical significance levels are for the difference between the Breathanol™ and placebo groups, with respect to the change in parameters after treatment.

TABLE VII

| | Before | After | | |
|---|---|---|---|---|
| | Treatment | Treatment | | Statistical |
| | (Active/ | (Active/ | | Significance |
| Parameter | Placebo) | Placebo) | Change | (p =) |
| Volatile | 3.68/3.67 | 3.60/3.84 | 0.08/−0.17 | 0.042 |
| Suiphide | | | | |
| Levels | | | | |
| Colour | 1.16/0.92 | 0.69/0.73 | 0.45/.0.19 | 0.37 |
| Test | | | | |
| Whole | 1.59/1.57 | 0.74/1.17 | 0.85/0.40 | 0.17 |
| mouth odor | | | | |
| Judge 1 | | | | |
| Whole | 2.12/2.10 | 1.90/1.51 | 0.18/0.59 | 0.19 |
| mouth odor | | | | |
| Judge 2 | | | | |
| Posterior | 1.96/1.92 | 1.97/1.87 | −0.001/0.05 | 0.77 |
| tongue odor | | | | |
| Judge 1 | | | | |
| Posterior | 2.80/2.75 | 2.35/2.63 | 0.45/0.12 | 0.28 |
| tongue odor | | | | |
| Judge 2 | | | | |

It may be seen from these results that there is a significant reduction in volatile sulphides (p=0.042) following use of the mouth drops containing Breathanol™, as compared with the placebo mouth drops.

### Example 5

### Inhibition of in vitro Odor Formation in Saliva by Lactones

The following five aroma flavors (obtained from Aromor Ltd) were tested for their ability to reduce odor formation in samples of saliva:

gamma-methyl decalactone

gamma-decalactone

gamma-undecalactone

gamma-dodecalactone

cis-3-hexenyl acetate

Morning saliva (0.2 ml) was added to 5 ml of decarboxylase medium. Following this, 0.03 ml of one of the aforementioned aroma flavors diluted to 5% (w/w) in food grade ethanol was added to the medium. The mixture was then incubated at 37° C. for 3 days. The odor of each sample was assessed by two odor judges using the odor scale described hereinabove, in Example 1. The results are shown in Table VIII.

TABLE VIII

| Aroma | Odor score |
|---|---|
| gamma-methyl decalactone | 3 |
| gamma-decalactone | 1 |
| gamma-undecalactone | 0 (pleasant odor) |
| gamma-dodecalactone | 2 |
| cis-3-hexenyl acetate | 2 (yeast-like odor) |

It may be seen from these results that of the agents tested, gamma-undecalactone was the most effective in preventing odor formation.

### Formulations

### Formulation 1

Toothpaste I

| | % (w/w) |
|---|---|
| MIX A: | |
| Sodium alginate | 1 |
| Calcium carbonate | 38 |
| Aerosil 2000 silica (Degusse) | 1.6 |
| Glycerin (86%) | 25 |
| Mineral oil DAB 10 | 0.5 |
| Sodium saccharin (Bayer) | 0.10 |
| Sodium monofluorophosphate | 0.76 |
| (Phoskadent NA 211, Benokiser) | |
| Breathanol ™ | 0.02-2.5 |
| MIX B: | |
| Sodium lauryl sulfate | 1.5 |
| Nipagin M preservative (Nipa) | 0.1 |
| Water | to 100% |

Preparation

1. The sodium alginate is added to the glycerin and allowed to swell.

2. The sodium lauryl sulfate is dissolved in approximately 5 parts water.

3. All the remaining ingredients are added.

4. The preparation is homogenized under vacuum.

To fully exploit the invention, the toothpaste should be brought into contact with the posterior region of the mouth by brushing the tongue, as well as with the teeth and gingivae, or alternatively by gargling.

### Formulation 2

Toothpaste II

| | % (w/w) |
|---|---|
| Keitrol (Kelco) Xanthan gum | 0.8 |
| Glycerin (86%) | 25 |
| Sorbitol (70%) | 15 |
| Sident 12DS Silica (Degusse) | 21 |
| Syloblanc 34 Silica (Grece) | 1 |
| Titanium dioxide | 1 |
| Sodium fluoride | 0.22 |
| Sodium saccharin | 0.1 |
| Breathanol ™ | 0.02-2.5 |
| Sodium Lauryl sulfate | 2 |
| Preservative | 0.1 |
| Water | to 100% |

Preparation

1. Swell the xanthan gum in the sorbitol and glycerin.

2. Add all other ingredients

3. Homogenize under vacuum.

To fully exploit the invention, the toothpaste should be brought into contact with the posterior region of the mouth by brushing the tongue, as well as with the teeth and gingivae, or alternatively by gargling.

### Formulation 3

Sugarless chewing gum

| | % (w/w) |
|---|---|
| Gum base (Jagum T) | 30 |
| Sorbitol (70%) | 14 |
| Glycerin | 1 |
| Sorbit powder | 40 |
| Palatinit | 9.8 |
| Mannitol | 3 |
| Xylitol | 2 |
| Aspartame | 0.1 |
| Acesulfam K | 0.1 |
| Breathanol ™ | 0.02-2.5 |

### Formulation 4

Hard candy

| | % (w/w) |
|---|---|
| Saccharose | 57 |
| Glucose syrup | 29 |
| Breathanol ™ | 0.02-2.5 |
| Water | to 100% |

Preparation

1. Dissolve the saccharose in the water at 110° C.

2. Add the glucose syrup and heat to 140° C.

3. Add the Breathanol™ and citric acid.

4. Pour into moulds at 130-135° C.

### Formulation 5

Soft candy

| | % (w/w) |
|---|---|
| Sucrose/refined batch I | 35 |
| Spray-sour whey powder | 1.1 |
| Water | 10.5 |
| Glucose syrup (38-40 DE) | 42 |
| Hard fat D 700 S (SP 34-36° C.) | 4.0 |
| Lecithin | 0.1 |
| Fudge mass | 5.8 |
| Gelatin (e.g., 230 Bloom) | 0.3 |
| Water (for swelling the gelatin) | 1.1 |
| Breathanol12 ™ | 0.02-2.5 |
| Water | to 100% |
| Preparation: | |

1. Dissolve Sucrose and Whey powder in water and boil until clear

2. At about 115° C. add glucose syrup and hard fat plus Lecithin and mix well

3. Boil the mass at 122° C.

4. Cool to about 90° C. and then add fudge and well dissolved gelatin (at temperatures of more than 90° C. the gelatin may be damaged)

Disperse the breathanol well into the mass.

5. Cool the mass on a precooled cooling table, allow equilibration of temperature, and then stretch until the desired consistency is reached, shape into desired form.

### Formulation 6

Two-phase mouth-spray/mouthwash

| | % (w/w) |
|---|---|
| 70% sorbitol | 10 |
| Sodium benzoate | 0.1 |
| Sodium saccharine | 0.05 |
| Breathanol | 0.02-2.5 |
| Mint oil | 0.2 |
| Oil | 15.0 |
| Water | to 100% |

To fully exploit the invention, the mouthwash should be gargled.

### Formulation 7

Single-phase mouth-spray/mouthwash

| | % (w/w) |
|---|---|
| 70% Sorbitol | 10 |
| Sodium benzoate | 0.1 |
| Sodium saccharine | 0.05 |
| Breathanol | 0.02-2.5 |
| Mint oil | 0.2 |
| Ethanol | 6 |
| Tagat RH40 (Tzifroni) | 2 |
| Water | to 100% |

To fully exploit the invention, the mouthwash should be gargled.

### Formulation 8

Mouth drops I

| | % (w/w) |
|---|---|
| CMC | 3 |
| 70% Sorbitol | 10 |
| Sodium benzoate | 0.1 |
| Sodium saccharine | 0.05 |
| Creamogen MZ | 0.2 |
| (H&R) | |
| Ethanol | 20 |
| Tagat RH40 (Tzifroni) | 2.1 |
| Breathanol | 0.2-2.5 |
| Mint oil | 0.2 |
| Water | to 100% |

### Formulation 9

Mouth drops II

| | % (w/w) |
|---|---|
| Glycerin (vegetable) | 3 |
| Capric/Caprylic | 12 |
| triglycerides | |
| (fractionated coconut | |
| oil) | |
| Sucrose esters of fatty | 1 |
| acids (E473) | |
| Breathanol | 1 |
| Water | to 100% |

In addition to liquid and semi-solid products (such as e.g., candies), the compositions of the invention can be used to coat or impregnate inert materials, such as toothpicks, dental floss and the like. Furthermore, the compositions of the invention can be administered in gaseous form, e.g., they can be evaporated from cigarettes.

All the above description of preferred embodiments have been provided for the purpose of illustration and are not intended to limit the invention in any way. Many modifications can be made to the compositions and methods, without exceeding the scope of the invention.

1. An oral anti-odor composition comprising a higher alcohol selected from 1-nonanol, 1-decanol and 1-undecanol, or mixtures thereof, and taste-masking additives.

2. A composition according to claim 1, wherein the higher alcohol is 1-nonanol.

3. A composition according to claim 1, wherein the higher alcohol is 1-decanol.

4. A composition according to claim 1, wherein the higher alcohol is 1-undecanol.

5. A composition according to any one of claims 1 to 4, wherein the taste-masking additives comprise nerol, citral and menthol, mint, spearmint and peppermint oils, or other mint flavors, or mixtures thereof.

6. A composition according to any one of claims 1 to 5, provided in a glycerol-based carrier.

7. An anti-odor toothpaste comprising a higher alcohol selected from 1-nonanol, 1-decanol and 1-undecanol, or mixtures thereof, together with one or more taste-masking additives.

8. An anti-odor toothpaste according to claim 7, wherein the higher alcohol is 1-nonanol.

9. An anti-odor toothpaste according to claim 7, wherein the higher alcohol is 1-decanol.

10. An anti-odor toothpaste according to claim 7, wherein the higher alcohol is 1-undecanol.

11. An anti-odor toothpaste according to any one of claims 7 to 10, wherein the taste-masking additives comprise nerol, citral and menthol, mint, spearmint and peppermint oils, or other mint flavors.

12. An anti-odor candy comprising a higher alcohol selected from 1-nonanol, 1-decanol and 1-undecanol, or mixtures thereof, together with one or more taste-masking additives.

13. An anti-odor candy according to claim 12, wherein the candy is a chocolate candy.

14. An anti-odor candy according to claim 12, wherein the candy is a chewable candy.

15. An anti-odor candy according to claim 12, wherein the candy is in the form of chewing gum.

16. A chewing gum according to claim 15, wherein the higher alcohol and the additives are incorporated in the gum, and the chewing gum is covered by a dragé.

17. An anti-odor candy according to claim 12, wherein the candy is a slowly dissolving product.

18. An anti-odor candy according to claim 17, wherein the slowly dissolving product is a pastille.

19. An anti-odor candy according to claim 17, wherein the slowly dissolving product is a hard candy.

20. An anti-odor candy according to any one of claims 12 to 19, wherein the higher alcohol is 1-nonanol.

21. An anti-odor candy according to any one of claims 12 to 19, wherein the higher alcohol is 1-decanol.

22. An anti-odor candy according to any one of claims 12 to 19, wherein the higher alcohol is 1-undecanol.

23. An anti-odor candy according to any one of claims 12 to 22, wherein the taste-masking additives comprise nerol, citral and menthol, mint, spearmint or peppermint oils, or other mint flavors.

24. An anti-odor mouthwash comprising a higher alcohol selected from 1-nonanol, 1-decanol and 1-undecanol, or mixtures thereof, together with taste-masking additives.

25. An anti-odor mouthwash according to claim 24, wherein the higher alcohol is 1-nonanol.

26. An anti-odor mouthwash according to claim 24, wherein the higher alcohol is 1-decanol.

27. An anti-odor mouthwash according to claim 24, wherein the higher alcohol is 1-undecanol.

28. An anti-odor mouthwash according to any one of claims 24 to 27, wherein the taste-masking additive comprises comprise nerol, citral and menthol, mint, spearmint or peppermint oils, or other mint flavors.

29. An anti-odor mouthspray comprising a higher alcohol selected from 1-nonanol, 1-decanol and 1-undecanol, or mixtures thereof, together with taste-masking additives.

30. Anti-odor mouthdrops comprising a higher alcohol selected from 1-nonanol, 1-decanol and 1-undecanol, or mixtures thereof, together with taste-masking additives.

31. An anti-odor cigarette, comprising a higher alcohol selected from 1-nonanol, 1-decanol and 1-undecanol, or mixtures thereof.

32. A toothpick coated or impregnated with a composition comprising a higher alcohol selected from among 1-nonanol, 1-decanol and 1-undecanol, or mixtures thereof, together with taste-masking additives.

33. A dental floss yarn coated or impregnated with a composition comprising a higher alcohol selected from among 1-nonanol, 1-decanol and 1-undecanol, or mixtures thereof, together with taste-masking additives.

34. An anti-odor composition adapted be brought into contact with the oro-pharynx and posterior portion of the oral cavity, comprising a higher alcohol selected from 1-nonanol, 1-decanol and 1-undecanol, or mixtures thereof, together with one or more taste-masking additives.

35. Anti-odor paste adapted to be applied to the tongue and to the teeth, comprising a higher alcohol selected from 1-nonanol, 1-decanol and 1-undecanol, or mixtures thereof, together with one or more taste-masking additives.

36. Anti-odor paste adapted to be supplied to the teeth and to be gargled, comprising a higher alcohol selected from 1-nonanol, 1-decanol and 1-undecanol, or mixtures thereof, together with one or more taste-masking additives.

37. An oral anti-odor composition comprising a lactone selected from gamma-methyl decalactone, gamma-decalactone, gamma-undecalactone and gamma-dodecalactone, or mixtures thereof.

38. An oral anti-odor composition according to claim 37, further comprising a higher alcohol selected from 1-nonanol, 1-decanol and 1-undecanol, or mixtures thereof.

39. An oral anti-odor composition according to claim 37 or claim 38, wherein the lactone is gamma-undecalactone.

40. Use of a lactone as a synergistic additive in an oral anti-odor composition.
